(19) United States
(12) Patent Application Publication
(10) Pub. No.: US 2026/0047923 A1
(43) Pub. Date: Feb. 19, 2026

(54) FLARED PROSTHETIC CARDIAC VALVE DELIVERY DEVICES AND SYSTEMS

(71) Applicant: Shifamed Holdings, LLC, Campbell, CA (US)

(72) Inventors: Ali SALAHIEH, Saratoga, CA (US); Connor Gordon MULCAHY, San Francisco, CA (US); Alice YANG, Campbell, CA (US); Savina BRACHTHAUSER BALCELLS, Los Gatos, CA (US); Mitchell YOUNG, San Francisco, CA (US); Jonathan Hwan OAKDEN, San Jose, CA (US); Jasper Ellington ADAMEK-BOWERS, San Francisco, CA (US); Cornelius Matthew CROWLEY, San Francisco, CA (US); Terrence VICK, Los Gatos, CA (US); Balvir JOHAL, San Jose, CA (US); Thu Hoang PHAM, San Jose, CA (US)

(21) Appl. No.: 19/217,801

(22) Filed: May 23, 2025

Related U.S. Application Data
(63) Continuation of application No. 17/543,555, filed on Dec. 6, 2021, now Pat. No. 12,329,635.
(60) Provisional application No. 63/121,812, filed on Dec. 4, 2020.
(60) Provisional application No. 63/173,281, filed on Apr. 9, 2021.
(60) Provisional application No. 63/274,821, filed on Nov. 2, 2021.

(51) Int. Cl. A61F 2/24

(52) U.S. Cl. CPC A61F 2/2418; A61F 2220/0008; A61F 2220/0025; A61F 2230/005; A61F 2230/0078; A61F 2230/0091; A61F 2250/0039

(57) ABSTRACT

A device for treating a diseased native valve in a patient is provided, the device including a frame structure; a valve segment positioned radially within the frame structure, the valve segment comprising a plurality of leaflets, and a plurality of commissure attachment mechanisms coupling the leaflets to the frame structure, each commissure attachment mechanism extending radially inwards from an outflow end of the frame structure as to create a gap between an interior diameter of the outflow end and an outflow edge of the valve segment. Other embodiments and methods of use are also provided.

### CROSS-REFERENCE TO RELATED APPLICATIONS

This application is a continuation application of U.S. patent application Ser. No. 17/543,555, filed Dec. 6, 2021, titled “FLARED PROSTHETIC CARDIAC VALVE DELIVERY DEVICES AND SYSTEMS”, which claims the benefit of U.S. Provisional Application No. 63/121,812, filed Dec. 4, 2020, titled “MINIMAL FRAME PROSTHETIC CARDIAC VALVE DELIVERY DEVICES, SYSTEMS, AND METHODS”, and to U.S. Provisional Application No. 63/173,281, filed Apr. 9, 2021, titled “FLARED PROSTHETIC CARDIAC VALVE DELIVERY DEVICES AND SYSTEMS”, and to U.S. Provisional Application No. 63/274,821, filed Nov. 2, 2021, titled “FLARED PROSTHETIC CARDIAC VALVE DELIVERY DEVICES AND SYSTEMS”, all of which are incorporated by reference as if fully set forth herein for all purposes.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve devices, for example, is that the mitral valve devices are too large in diameter to be delivered transeptally, requiring transapical access instead. Furthermore, existing mitral valve replacement devices are not optimized with respect to strength-weight ratio and often take up too much space within the valve chambers, resulting in obstruction of outflow from the ventricle into the aorta and/or thrombosis.

Thus, a new valve device that overcomes some or all of these deficiencies is desired.

### SUMMARY OF THE DISCLOSURE

A device for treating a diseased native valve in a patient is provided, the device comprising a frame structure; a valve segment positioned radially within the frame structure, the valve segment comprising a plurality of leaflets, and a plurality of commissure attachment mechanisms coupling the leaflets to the frame structure, each commissure attachment mechanism extending radially inwards from an outflow end of the frame structure as to create a gap between an interior diameter of the outflow end and an outflow edge of the valve segment.

In some embodiments, an inflow edge of the valve segment is unsupported by the frame structure. In other embodiments, the inflow edge is spaced radially inwards from an inflow end of the frame structure. In some embodiments, an inflow end of the frame structure is flared radially outwards.

In one embodiment, the outflow end of the frame structure is flared radially outwards, and wherein tips of the outflow end point substantially axially.

In some embodiments, the commissure attachment mechanisms each comprise a paddle, the paddle including a slot therein through which tabs of the leaflet commissures pass.

In one example, the paddle further comprises a plurality of holes therethrough for sewing attachment of the tabs to the paddle.

In another embodiment, the commissure attachment mechanisms each include a post that attaches to the outflow end of the frame structure and curves radially inwards.

In some examples the post curves in approximately 180 degrees.

In some embodiments, the post attaches to a strut of the outflow end, and wherein a thickness of the post is greater than a thickness of the strut.

In one embodiment, the gap is between 1.5 mm and 4 mm when the leaflets are fully opened.

In some examples, the leaflets are unsupported except at the commissure attachment mechanisms.

In some embodiments, the device further comprises a spiral anchor configured to be placed around the frame structure.

In some embodiments, the frame structure comprises a plurality of struts, and wherein the struts have a narrowed portion proximate to the spiral anchor.

A device for treating a diseased native valve in a patient is also provided, the device comprising a frame structure comprising a central annular portion, an inflow portion, and an outflow portion, wherein the outflow portion is flared radially outwards relative to the central annular portion; a valve segment positioned radially within the frame structure, the valve segment comprising a plurality of leaflets, and a plurality of commissure attachment mechanisms coupling the leaflets to the outflow portion of the frame structure, each commissure attachment mechanism extending radially inwards such that an inner circumference formed by the commissure attachment mechanisms is approximately equal to an inner circumference of the central annular portion.

In some embodiments, an inflow edge of the valve segment is unsupported by the frame structure. In other embodiments, the inflow edge is spaced radially inwards from the inflow portion of the frame structure. In some examples, the inflow portion of the frame structure is flared radially outwards.

In some embodiments, tips of the outflow portion point substantially axially. In one embodiment, the commissure attachment mechanisms each comprise a paddle, the paddle including a slot therein through which tabs of the leaflet commissures pass.

In another embodiment, the paddle further comprises a plurality of holes therethrough for sewing attachment of the tabs to the paddle.

In some examples, the commissure attachment mechanisms each include a post that attaches to the outflow portion of the frame structure and curves radially inwards.

In one embodiment, the post curves in approximately 180 degrees. In other examples, the post attaches to a strut of the outflow portion, and wherein a thickness of the post is greater than a thickness of the strut.

In one embodiment, a gap between an outflow edge of the valve segment and an inner perimeter (e.g., diameter) of the outflow portion of the frame structure is between 1.5 mm and 4 mm when the leaflets are fully opened.

In some examples, the leaflets are unsupported except at the commissure attachment mechanisms.

In one embodiment, the device further comprises a spiral anchor configured to be placed around the frame structure at the central annular portion.

In some examples, the frame structure comprises a plurality of struts, and wherein the struts have a narrowed portion within the central annular portion.

A device for treating a diseased native valve in a patient is provided, the device comprising a frame structure comprising an annular central portion, a flared inflow portion, and a flared outflow portion, a valve segment positioned radially within the frame structure, the valve segment comprising a plurality of leaflets, and an internal skirt attached to the frame structure, the internal skirt comprising a plurality of convex segments configured to at least partially conform to inflow edges of the leaflets.

In some embodiments, the convex segments have a greater radius of curvature than the inflow edges of the leaflets.

In one example, an outflow edge of the internal skirt comprises a zig-zag pattern configured to match a cell pattern of the frame structure. In other examples, an outflow edge of the internal skirt is attached to the frame proximate to the annular central portion. In another embodiment, an outflow edge of the internal skirt does not extend to an outflow end of the frame structure.

In some examples, the inflow edges of the leaflets are unsupported by the frame structure. In another embodiment, the inflow edges of the leaflets are spaced radially inwards from an inflow end of the frame structure.

In some embodiments, the device further comprises a spiral anchor configured to be placed around the frame structure.

A device for treating a diseased native valve in a patient is provided, the device comprising a frame structure comprising an annular central portion, a flared inflow portion, and a flared outflow portion; a valve segment positioned radially within the frame structure, the valve segment comprising a plurality of leaflets, and an external skirt attached to the frame structure, the external skirt comprising a unitary structure covering the flared inflow portion and the flared outflow portion.

In some examples, the external skirt comprises a tube knit fabric. In another embodiment, the external skirt comprises a coating thereon.

In some embodiments, the device further comprises a spiral anchor configured to be placed around the frame structure.

In one embodiment, the external skirt is wrapped over an outflow edge of the frame structure.

In another embodiment, an additional skirt layered with the external skirt. In some examples, the additional skirt is positioned along the flared outflow portion. In one embodiment, the additional skirt is positioned along a central annular portion of the frame structure.

A device for treating a diseased native valve in a patient is provided, the device comprising a frame structure comprising a flared inflow portion comprising first and second rows of cells; an annular central portion comprising a third row of cells, and a flared outflow portion comprising a fourth row of cells, and a valve segment positioned radially within the frame structure, the valve segment comprising a plurality of leaflets.

In some embodiments, the inflow portion is flared further radially outwards than the outflow portion.

In other embodiments, the inflow portion is curved so as to point radially inwards.

In one embodiment, the device further comprises a plurality of non-foreshortening elements extending from the inflow portion.

In some examples, tips of the outflow portion point substantially axially.

In one embodiment, the third row of cells comprises a plurality of narrowed axially extending struts therein.

In other embodiments, tips of the flared outflow portion point substantially axially.

In some examples, the cells are substantially diamond-shaped.

In another embodiment, the device comprises a spiral anchor configured to be placed around the frame structure at the central annular portion.

In another example, the device includes a plurality of commissure attachment mechanisms coupling the leaflets to the frame structure, each commissure attachment mechanism extending radially inwards from an outflow end of the frame structure as to create a gap between an interior diameter of the outflow end and an outflow edge of the valve segment.

A method for treating a diseased native valve in a patient is provided, comprising: providing a device including a frame structure, a valve segment positioned radially within the frame structure, the valve segment comprising a plurality of leaflets, and a plurality of commissure attachment mechanisms coupling the leaflets to the frame structure; advancing the device to the diseased native valve in the patient; deploying the device within the diseased native valve to secure the device to the diseased native valve; and allowing an outflow edge of the plurality of leaflets to open to a radius that is greater than a radius formed by the plurality of commissure attachment mechanisms.

In some embodiments, the outflow edge of the plurality of leaflets is allowed to open to a radius that is less than an inner perimeter of the frame structure.

In other embodiments, the opening of the plurality of leaflets is configured to maintain a cylindrical flow path.

A method for treating a diseased native valve in a patient is provided, comprising: providing a device including a frame structure, a valve segment positioned radially within the frame structure, the valve segment comprising a plurality of leaflets, and a plurality of commissure attachment mechanisms coupling the leaflets to the frame structure, wherein the plurality of commissure attachment mechanisms extend radially inwards such that an inner circumference formed by the commissure attachment mechanisms is less than an inner circumference formed by the frame structure; advancing the device to the diseased native valve in the patient; deploying the device within the diseased native valve to secure the device to the diseased native valve; and allowing an outflow edge of the plurality of leaflets to open to a radius that is greater than a radius formed by the plurality of commissure attachment mechanisms.

In some embodiments, the outflow edge of the plurality of leaflets is allowed to open to a radius that is less than an inner perimeter of the frame structure.

In other embodiments, the opening of the plurality of leaflets is configured to maintain a cylindrical flow path.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIGS. 1A-1B show an implantable valve prosthesis, in accordance with embodiments.

FIG. 2 shows a frame structure of a valve prosthesis in a partial hourglass shape.

FIG. 3 shows a valve prosthesis in an unexpanded (or collapsed or crimped) configuration.

FIG. 4 shows a representative example an anchor for a valve prosthesis.

FIG. 5 is an example of a frame structure with one or more leaflets.

FIGS. 6-7 show a frame with a valve segment attached to one or more struts of the frame structure.

FIGS. 8A-8B show another valve prosthesis where substantially all of the inflow edge extends proximally beyond the proximal arches of the frame structure.

FIG. 9 shows another embodiment of a frame structure.

FIGS. 10A-10B show a valve prosthesis wherein the inflow edge of the valve segment is completely unsupported.

FIGS. 11A-11C show another valve prosthesis wherein the inflow edge of the valve segment is completely unsupported.

FIGS. 12A-12F show a frame structure including a close-up of commissure attachment mechanisms.

FIGS. 13A-13E illustrate one example of leaflets attached to the frame structure.

FIGS. 14A-14C illustrate the leaflets closing.

FIGS. 15A-15C illustrate the leaflets opening.

FIGS. 16A-16D illustrate various views of a frame structure with leaflets including dimensions.

FIGS. 17A-17D illustrate one example of a frame structure.

FIGS. 18A-18D illustrate another example of a frame structure.

FIGS. 19A-19D illustrate one example of a frame structure.

FIGS. 20A-20D illustrate yet another example of a frame structure.

FIG. 21 is a table showing exemplary dimensions for various frame structures.

FIGS. 22A-22B illustrate one example of a frame structure.

FIGS. 23A-23D illustrate another example of a frame structure.

FIGS. 24A-24D illustrate one example of a frame structure.

FIGS. 25A-25B illustrate another example of a frame structure.

FIGS. 26A-26C illustrate an internal skirt attached to a frame.

FIGS. 27A-27F illustrate attachment or sewing points of a skirt to the frame.

FIGS. 28-29 illustrate a frame with a unitary skirt.

FIGS. 30A-30B illustrate a frame with an internal skirt or an external skirt.

### DETAILED DESCRIPTION

Described herein are systems, devices, and methods for treatment or replacement of a diseased native valve of the heart, such as the mitral valve.

In general, described herein is a replacement prosthesis that can include a valve frame and a spiral anchor therearound.

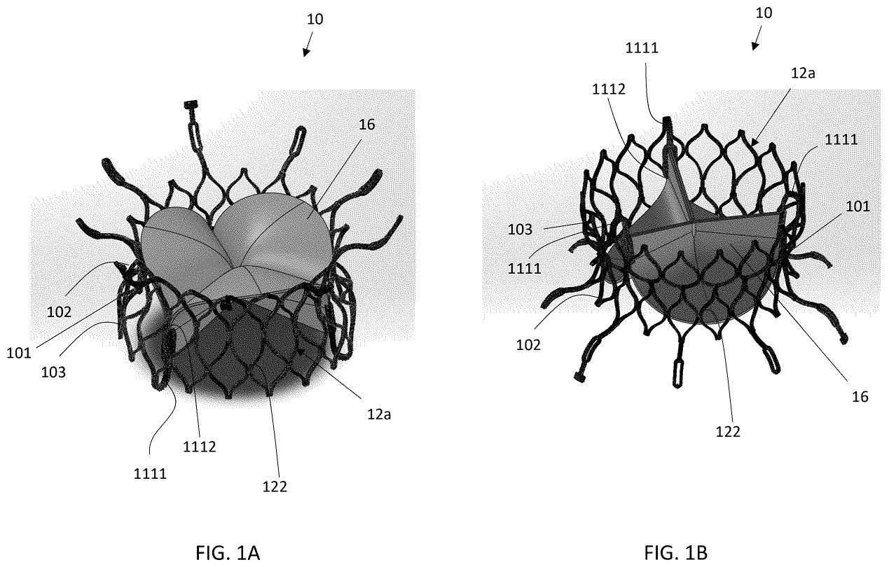

FIGS. 1A-1B show a valve prosthesis 10 having a valve frame 12a and a plurality of leaflets 16 therein. The valve frame 12a can include interior commissure attachment mechanisms 1111 for attachment of the commissures 1112 of leaflets 16 to the frame structure 12. The valve frame 12a can be deployed from a collapsed (delivery) configuration to an expanded configuration during a method of replacing or repairing a native valve, such as a mitral valve. As shown in FIGS. 1A-1B, the valve frame 12a can include a plurality of rows (e.g., 3-7 rows) of substantially diamond-shaped cells 122. The valve frame 12a can foreshorten during delivery (i.e., as the valve frame 12 transitions from the collapsed configuration to the expanded configuration) due to the cell structure. In some embodiments, the valve frame 12a can be configured to self-expand from the collapsed configuration to the expanded configuration (e.g., can be made of nitinol). The valve frame 12a can provide circumferential strength and/or longitudinal strength to valve prosthesis 10.

The valve prosthesis 10 can be deployed in an expanded configuration according to the methods described herein. For example, valve prosthesis 10 can be deployed into an expanded configuration in a method of replacing or repairing a native anatomical structure. In the expanded configuration, valve prosthesis 10 can be positioned and/or anchored at a target region of a subject (e.g., an organ or tissue of an animal such as a dog, cat, horse, or human). For example, valve prosthesis 10 can be positioned in the expanded configuration in the orifice of a heart valve, such as the mitral valve or tricuspid valve (e.g., to function as a temporary or permanent replacement for an existing mitral valve or tricuspid valve of the heart).

One or more portions of the valve frame 12a can be shaped or configured to aid in securing the valve frame 12 at a location (e.g., in the orifice of a native heart valve). For example, the valve frame 12a can include an atrial flared portion 102 and a ventricular flared portion 103 configured to help secure the frame in the anatomy. The atrial and ventricular flared portions 102, 103 can extend radially outwards from a central circumferential portion 101. The atrial flared portion 102 can, for example, extend into the atrium of the heart from the central circumferential portion 101 when the valve prosthesis is deployed in the native mitral valve. The ventricular flared portion 103, in turn, can extend into the ventricle of the heart from the central circumferential portion 101 when the valve prosthesis is deployed in the native mitral valve. The atrial and ventricular flared portions 102, 103 can, for example, be configured to be positioned on either side of an external flat spiral anchor (e.g., that is wrapped around the chordae) to anchor the valve frame 12 in the anatomy. Alternatively or additionally, the atrial and ventricular flared portions 102, 103 can be configured to engage with tissue to prevent the valve prosthesis from slipping through the native valve orifice.

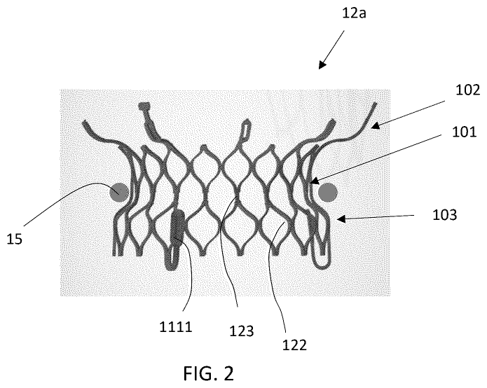

Referring to FIG. 2, the frame structure 12a of the valve prosthesis 10 can be in a partial hourglass shape such that the flared ventricular portion 103 initially flares radially outwards, but then curves to point substantially axially (i.e., in the ventricular direction). This half-hourglass or cup shape of the ventricular portion 103 can advantageously help provide space for the chordae therearound.

As shown in FIG. 2, the spacing between the atrial and ventricular portions 102, 103 (i.e., along the smaller diameter central annular portion 101) can enable the anchor 15 to lodge therebetween to better anchor the prosthesis in place in the native valve. Further, as shown in FIG. 2, the annular central portion 101 can include axially extending struts 123 that are narrower than the struts that form the rest of the diamond-shaped cells 122. The narrowed axially extending struts 123 can create an area of flexibility within the annular central portion 101, encouraging the anchor 15 to rest against that portion of the frame structure 12a.

Further, as shown in FIGS. 1A-1B and 2, the atrial flared portion 102 can extend further radially outwards than the ventricular flared portion 103. Having a larger atrial flared portion can help prevent para-valvular leakage (PVL).

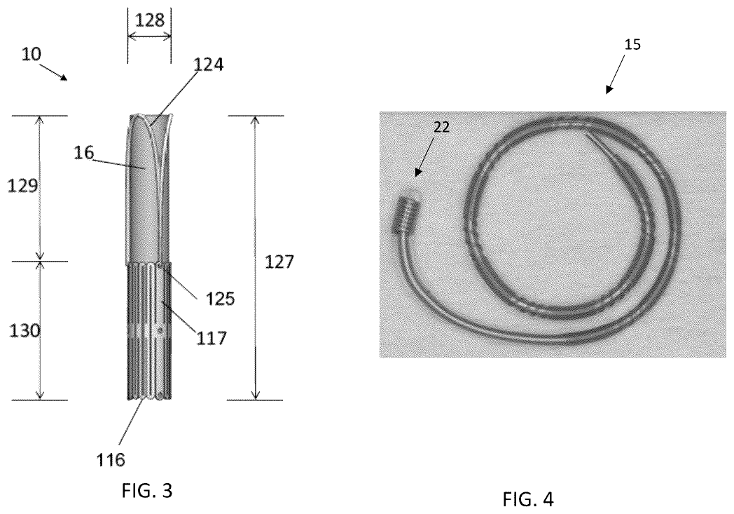

FIG. 3 shows a valve prosthesis in an unexpanded (or collapsed or crimped) configuration. In some cases, the valve prosthesis 10 can be delivered to a target region (e.g., a region of a heart comprising a native valve) in the unexpanded configuration. In some cases, the valve prosthesis 10 in the unexpanded configuration can allow the valve prosthesis 10 to be delivered via minimally invasive means (e.g., via a delivery device, as described herein).

In some embodiments, the longitudinal length 127 of the collapsed valve frame 12a can be minimized, which can be advantageous for delivery of the valve frame 12a. For example, minimizing the overall longitudinal length of the collapsed valve frame 12a can allow improved maneuverability within a delivery device while maintaining structural strength of the device. In some embodiments, minimizing the overall longitudinal length of the collapsed valve frame 12a can allow insertion of valve frame 12a through an access path that would be challenging for a longer device to traverse (e.g., an access path comprising tortuous passages or passages with sharp turns). In some embodiments, the valve frame 12a in the unexpanded configuration can have an overall longitudinal length of from 1 mm to 50 mm, from 1 mm to 45 mm, from 1 mm to 40 mm, from 1 mm to 35 mm, from 1 mm to 30 mm, from 1 mm to 25 mm, from 1 mm to 20 mm, from 1 mm to 10 mm, from 10 mm to 45 mm, from 20 mm to 45 mm, from 20 mm to 30 mm, from 25 mm to 35 mm, or from 27.5 mm to 32.5 mm. In some embodiments, the valve frame 12a in the expanded configuration can have an overall longitudinal length of from 1 mm to 45 mm, from 10 mm to 45 mm, from 15 mm to 45 mm, from 15 mm to 35 mm, from 16 mm to 34 mm, from 17 mm to 33 mm, from 18 mm to 32 mm, from 19 mm to 31 mm, from 20 mm to 30 mm, from 25 mm to 35 mm, or from 27.5 mm to 32.5 mm. In some embodiments, the valve frame 12a can foreshorten as it expands such that the length in the expanded configuration is less than the length in the collapsed configuration.

In some embodiments, the valve frame 12a and/or overall prosthesis can have specific features designed to increase stiffness, improve control over valve deployment, promote uniform radial expansion of the central circumferential portion, ensure anchoring within the annulus, and/or decrease PVL.

Further, the diameter 128 of the collapsed valve prosthesis 10 can be minimized, which can likewise be advantageous for delivery of the valve prosthesis 10. For example, a collapsed valve prosthesis 10 with a smaller diameter 128 can fit inside of a delivery device with a smaller diameter, allowing for less invasive delivery and for improved maneuvering capability inside of a subject's body. Reducing the diameter 128 of the collapsed valve prosthesis 10 (e.g., for use in treatment or replacement of a mitral valve, a tricuspid valve, an aortic valve, or a pulmonic valve) can further allow for easier delivery of the valve prosthesis 10 to a target region of a subject, faster recovery of a subject receiving valve prosthesis 10, and/or improved clinical outcomes for a subject receiving valve prosthesis 10 (e.g., improved subject survival, improved ejection fraction, improved cardiac output, decreased valvular regurgitation, and/or decreased edema). In some cases, reducing the diameter 128 of the collapsed valve prosthesis 10 can make transseptal access and delivery possible in addition to transapical access. In some cases, the diameter 128 of the collapsed valve prosthesis 10 or portion thereof (e.g., frame structure 12) can be from 0.01 mm to 20 mm, 0.01 mm to 15 mm, 0.01 mm to 10 mm, from 0.01 mm to 9 mm, from 0.01 mm to 8 mm, from 0.01 mm to 7 mm, from 0.01 mm to 6 mm, from 0.01 mm to 5 mm, from 0.01 mm to 4 mm, from 0.01 mm to 3 mm, from 0.01 mm to 2 mm, from 0.01 mm to 1 mm, from 1 mm to 15 mm, from 2 mm to 14 mm, from 3 mm to 13 mm, from 4 mm to 12 mm, from 5 mm to 10 mm, from 6 mm to 10 mm, from 7 mm to 10 mm, from 8 mm to 10 mm, from 9 mm to 10 mm, from 10 mm to 15 mm, no more than 20 mm, no more than 15 mm, no more than 10 mm, no more than 9 mm, no more than 8 mm, no more than 7 mm, no more than 6 mm, or no more than 5 mm.

In some cases, the valve prosthesis 10 or a portion thereof can be sized or shaped to be positioned at a certain location or target region. For example, the frame structure 12 can be sized to be positioned in a valve, such as the mitral valve (e.g., by designing a dimension of frame structure to fit a valve, such as the mitral valve, when in an expanded configuration).

FIG. 4 shows a representative example of the an anchor 15 configured to position or anchor a valve prosthesis within a certain location such as a native valve. In some embodiments, the anchor 15 may comprise a flat shape that, for example, span around the valve prosthesis 10 in the unexpanded and/or expanded configuration. The anchor 15 can have a free end 22. In some cases, the free end 22 of anchor 15 can be useful during deployment of the anchor 15 in a native heart valve (e.g., by ensnaring chordae or other structures when the prosthesis 10, anchor 15, and/or delivery device are rotated around longitudinal axis of the valve prosthesis 10). The anchor 15 may be directly coupled to the frame structure 12a, for example at a first end (e.g., a proximal end) or a second end (e.g., a distal end) thereof. Alternatively, the anchor 15 can be physically uncoupled from the frame structure 12 while providing an anchor for the frame 12 as the frame expands within the native valve orifice (thereby sandwiching tissue between the frame 12 and the anchor 15). In some embodiments, the frame structure 12a can be at least partially held in place within the native valve via interaction with the anchor 15. For example, the expanded diameter of the frame structure 12a can be greater than or equal to the inner diameter of the spiraled anchor 15 such that the frame structure 12a expands into and engages with the anchor 15 (with native valve leaflets, chordae, or other tissue therebetween).

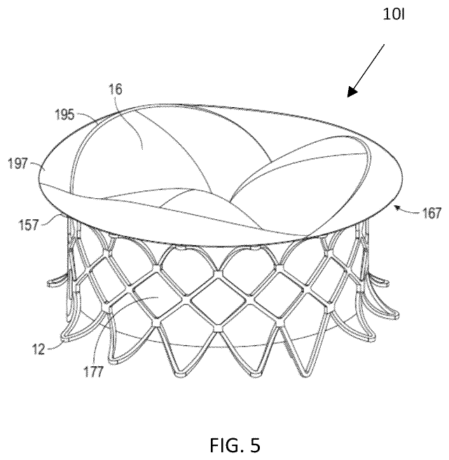

In some embodiments, the valve prostheses 10 described herein can include one or more flared portions to engage with the anchor 15 and/or help prevent the valve prostheses 10 from sliding through a valve orifice. For example, as shown in FIG. 5, the frame structure 12a of valve prosthesis 10I can include an atrial flared portion 157 extending radially outwards from a central annular portion 158. The atrial flared portion 157 can, for example, extend into the atrium of the heart from the central annular portion 158 when valve prosthesis 10A is deployed in a native mitral valve. Alternatively, or in combination, the atrial flared portion 157 can contact a tissue of the atrium of the heart, e.g., a mitral valve annulus when valve prosthesis 10A is deployed in a native mitral valve.

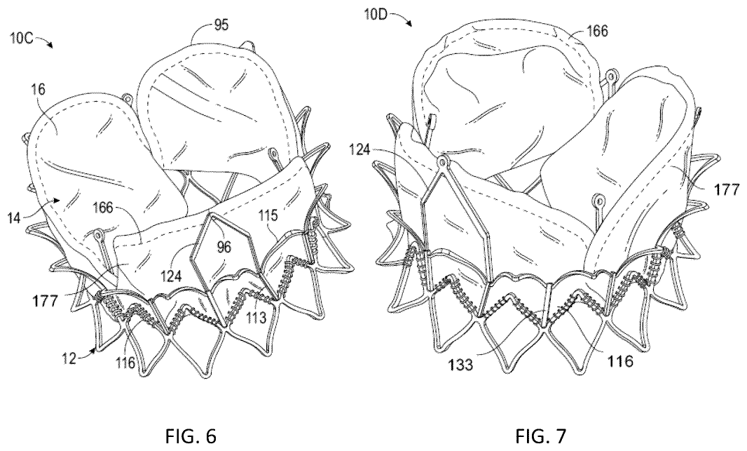

Referring to FIGS. 5-6, the valve prostheses 10 described herein may include one or more valve segments 14 disposed therein to replace the native valve leaflets. For example, the valve segment 14 can include a plurality of leaflets 16, e.g., that form a biocompatible one-way valve. Flow in one direction may cause the leaflets 16 to deflect open and flow in the opposite direction may cause the leaflets 16 to close.

Any of the valve segments 14 described herein may be formed of multi-layered materials for preferential function. Referring to FIG. 6, for example, the valve prosthesis 10C may include a valve segment 14 having a seal 177 (also called an outer leaflet, outer layer, or skirt) positioned radially between leaflets 16 (also called inner leaflets or the inner layer) and the frame structure 12. The seal 177 can be a single piece wrapped around the leaflets 16 or can be individual pieces shaped to match the leaflets 16. In some cases, the seal 177 and/or leaflets 16 can be formed from or coated with a material to confer an advantage upon the valve segment 14. For example, a layer or surface of a valve segment 14 can be formed from or coated with a biocompatible material. In some cases, a layer or surface of a valve segment 14 can be formed from or coated with an anti-thrombotic material. In some cases, a valve segment 14 (or portion thereof, such as a leaflet 16 of the valve segment) comprises a synthetic material. In some cases, a valve segment 14 (or portion thereof, such as a leaflet) comprises a biological tissue. In many cases, a valve segment 14 (or portion thereof, such as a leaflet) comprises pericardial tissue. In some embodiments, a valve segment 14 (or portion thereof, such as a leaflet 16 of the valve segment 14) comprises a decellularized biological tissue. For example, a valve segment 14 (or portion thereof, such as a leaflet 16 of the valve segment) can include decellularized pericardium.

The valve segment 14 may be attached to a frame structure 12, which can in turn be attached to the anchor 15. The frame structure 12 may be connected to the anchor 15 before or after the frame structure 12 has been deployed adjacent a native valve. The frame structure 12 may be attached to the valve segment 12, for example, via attachment of the frame structure 12 to the seal 177, which can in turn be attached to the leaflets 16.

In some embodiments, two or more portions of a valve segment 15 (e.g., two or more leaflets 16, and/or seal 177) can comprise a single piece of material (e.g., a single piece of biological or synthetic tissue formed into the shape of a functional valve). In some cases, two or more portions of a valve segment (e.g., two or more of a first and second leaflet 16, and/or the seal 177) can be joined together. In some embodiments, two or more portions of a valve segment (e.g., two or more of a first and second leaflet 16, and/or the seal 177) can be joined together by suturing the two or more portions together (e.g., at sutured coupling 166 shown in FIG. 6). In some cases, 1, 2, 3, 4, 5, or more than 5 leaflets 16 can be coupled to a single seal 177.

In many cases, leaflet coupling 166 is disposed at an inflow end of valve prosthesis 10 (i.e., closest to the source of flow through the device, e.g., caused by a contracting heart chamber) when deployed. In some cases, coupling two or more portions of a valve segment 14 at the inflow end of valve prosthesis 10 (or portion thereof) allows the valve segment 14 to fold or collapse (e.g., radially away from a longitudinal axis of valve prosthesis device 10) during contraction of a heart chamber upstream of the deployed device (i.e., during diastole). Further, in some cases, coupling two or more portions of a valve segment 14 at the inflow end of valve prosthesis 10 causes the valve segment 14 to expand (e.g., radially toward a longitudinal axis of valve prosthesis device 10) during refilling of a heart chamber upstream of the deployed device (i.e., during systole). This expansion of the valve segment 14 can, for example, result in billowing or parachuting of the valve segment 14 (e.g., between the seal 177 and the leaflets 16) to block the flow of blood therethrough.

As shown in FIG. 6, the valve segment 14 can be attached to one or more struts 113 of the frame structure 12. In some embodiments, a portion of a valve segment 14 (e.g., leaflets 16 or seal 177) can be sutured to the central annular portion 158 of frame structure 12 and not to the inflow portion of frame structure 12 or the outflow portion of frame structure 12 (e.g., can be unattached to the distal arches 116 and the proximal arches 115 as shown in FIG. 6).

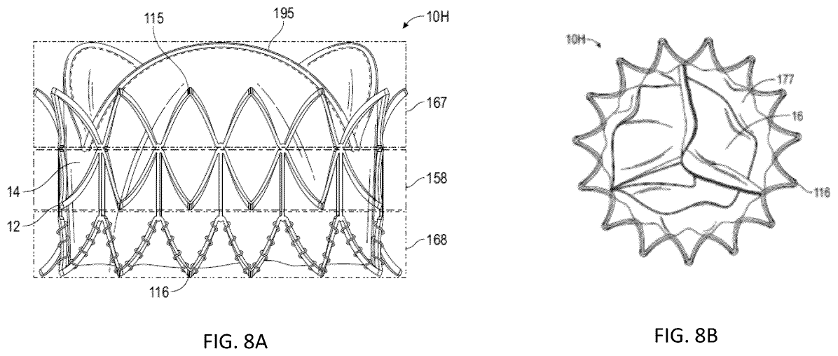

In some embodiments, a portion of a valve segment 14 (e.g., leaflets 16 or seal 177) can be sutured to one or more outflow portion of frame structure 12 and not to the inflow portion of frame structure 12 (e.g., can be sutured to one or more distal arches 116 but not one or more proximal arches 115 as shown in FIG. 8A).

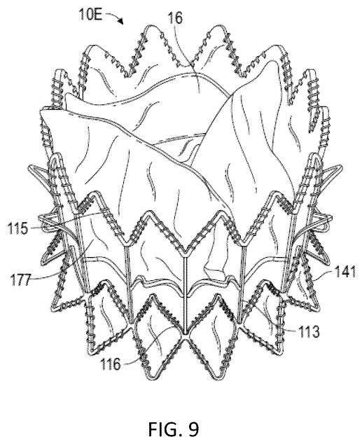

In some embodiments, a portion of the valve segment 14 can be sutured or otherwise attached with an outflow attachment mechanism (e.g., with inwardly extending commissure attachment mechanisms as illustrated and described further herein) and not to the inflow portion of the frame. In some embodiments, a portion of a valve segment 14 (e.g., leaflets 16 or seal 177) can be sutured to one or more outflow portion of frame structure 12 and to the inflow portion of frame structure 12 (e.g., can be sutured to one or more distal arches 116 and also to one or more proximal arches 115 as shown in valve prosthesis 10E of FIG. 9). In some embodiments, an inflow end of the valve segment 14 can be substantially unsupported by the frame 12 while the outflow end of the valve segment 14 can be fully supported by and within the valve segment 14 (as shown in FIG. 6). The valve segment 14 (or portion thereof, such as the seal 177) can be coupled to the frame 12 continuously around the inner circumference of the frame 12 (e.g., at a distal or outflow end of valve prosthesis device 10).

In some cases, the amount of attachment of a valve segment 14 (e.g., a valve leaflet 16) to the frame structure 12 can be minimized, which can advantageously enhance ease of delivery and reduce the required length of the frame, thereby reducing the chance of thrombosis and reducing the chance of blocking the outflow from the ventricle to the aorta. Minimizing the frame structure 12 can also improve the speed and cost of fabrication of the valve prosthesis device 10.

In some embodiments, a leaflet 16 that is attached to a first portion of frame structure 12 (e.g., one or more struts 113) at a distal end of frame structure 12 can be unattached at a proximal end of the frame structure 12 (e.g., a strut or portion thereof at a proximal end of frame structure 12). In some cases, valve prosthesis devices 10 in which a valve segment 14 is attached at a proximal end of frame structure 12 and is unattached at a proximal end of frame structure 12 (and/or at a proximal end of valve segment 14) may require less metal and/or fewer struts than a valve prosthesis 10 in which a valve segment 14 is attached at both a proximal end and a distal end of the frame structure 12 of the valve prosthesis device 10. In some cases, minimizing the amount of metal used in the structure of valve prosthesis 10 (e.g., by reducing the number and/or length of struts in valve prosthesis device 10) can reduce the risk of thrombus formation and can improve the ease with which the device is deployed at a target location.

Further, the valve segment 14 can be configured to be substantially unsupported at the inflow edge 95 of the valve segment 14. For example, as shown in FIG. 6, the entire inflow edge 95 of valve segment can be unsupported with the exception of minimal valve supports 124 positioned at the nadir 96 of each leaflet 16. The valve supports 124 can have a pointed proximal tip and can extend, for example, from two neighboring struts 113 of the frame structure 12. The minimal valve supports 124 can help prevent the valve segment 14 (e.g., the seal) from collapsing radially inwards in the outflow direction (i.e., towards the ventricle) when implanted in the heart. FIG. 7 shows a valve prosthesis 10D that is similar to valve prosthesis 10C of FIG. 4 except that the valve support 124 of FIG. 5 includes an aperture 97 for suturing the leaflet 16 to the valve support 124.

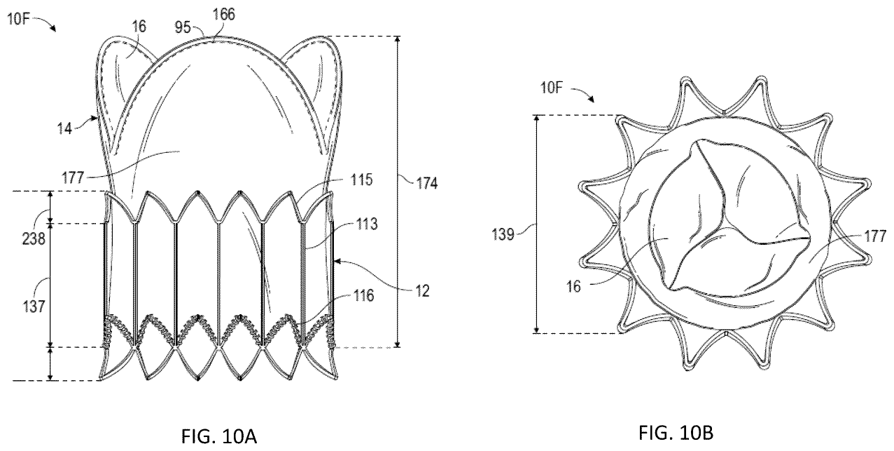

FIGS. 10A-10B show a valve prosthesis 10F wherein the inflow edge 95 of valve segment 14 is completely unsupported (i.e., does not include any valve supports thereto).

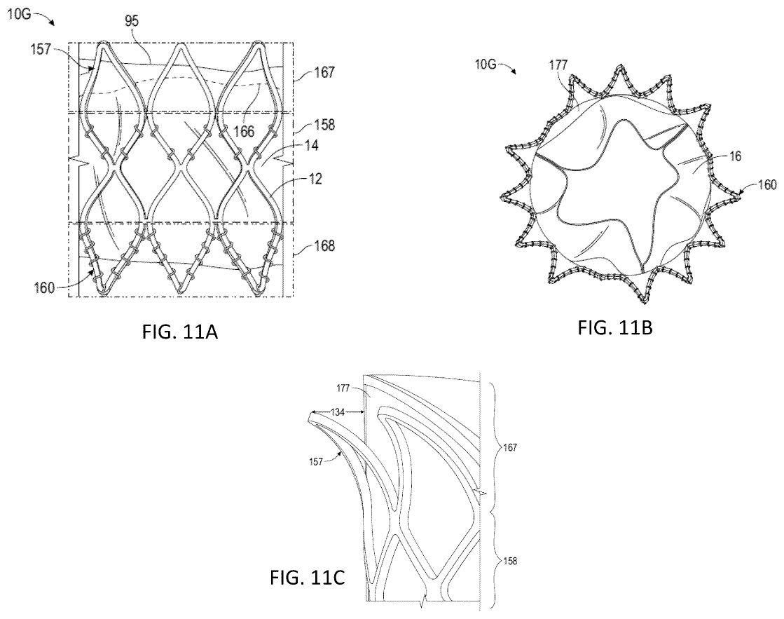

FIGS. 11A-11C show another valve prosthesis 10G wherein the inflow edge 95 of valve segment 14 is completely unsupported (i.e., does not include any valve supports thereto). Indeed, as shown in FIGS. 11A-11C, the prosthesis 10G can include an inflow portion 167, a central annular portion 158, and an outflow portion 168. The valve segment 14 can be fully circumferentially supported by the frame structure 12 within the central annular section 158. However, the valve segment 14 can be unsupported by and/or unconnected from the frame structure 12 in the inflow section 167. Further, the frame structure 12 can flare radially outwards within the inflow section 167. The flared portion 157 of the frame structure 12 can include a plurality of discrete flanges (i.e., formed from flared proximal arches 115) and can, for example, serve to help engage with an external anchor. Moreover, due to the flared portion 157, the valve segment 14 can be radially spaced away from the frame structure 12 within the inflow section 167 by a distance 134 (see FIG. 11C). In some embodiments, the distance 134 can be 1-10 mm, such as 2-8 mm, such as 3-5 mm. Finally, the frame structure 12 can also flare radially outwards within the outflow section 168. The flared portion 160 of the frame structure 12 can also serve to help engage with an external anchor 15. For example, the external anchor 15 can sit between the flared portions 157, 160 upon implantation.

FIGS. 8A-8B show another valve prosthesis 10H that is similar to valve prosthesis 10G of FIGS. 11A-11C except that substantially all of the inflow edge 95 extends proximally beyond the proximal arches 115 of the frame structure 12. When the leaflets are closed (as shown in FIG. 8B), the fluid pressure can act to fill the space created by the leaflets 16 and the seal 177, thereby preventing inward motion or collapse of the valve segment 14.

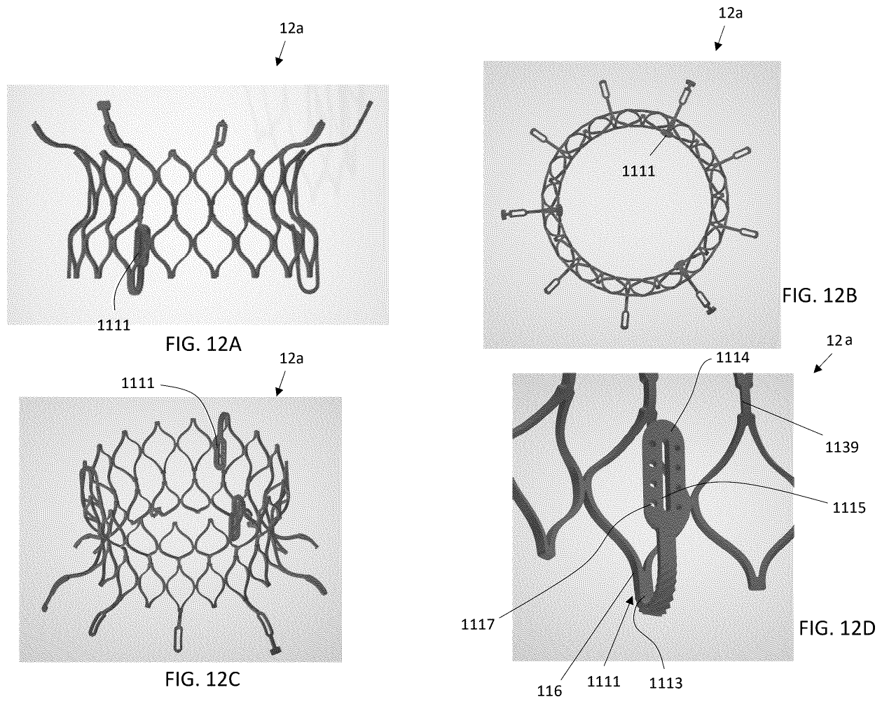

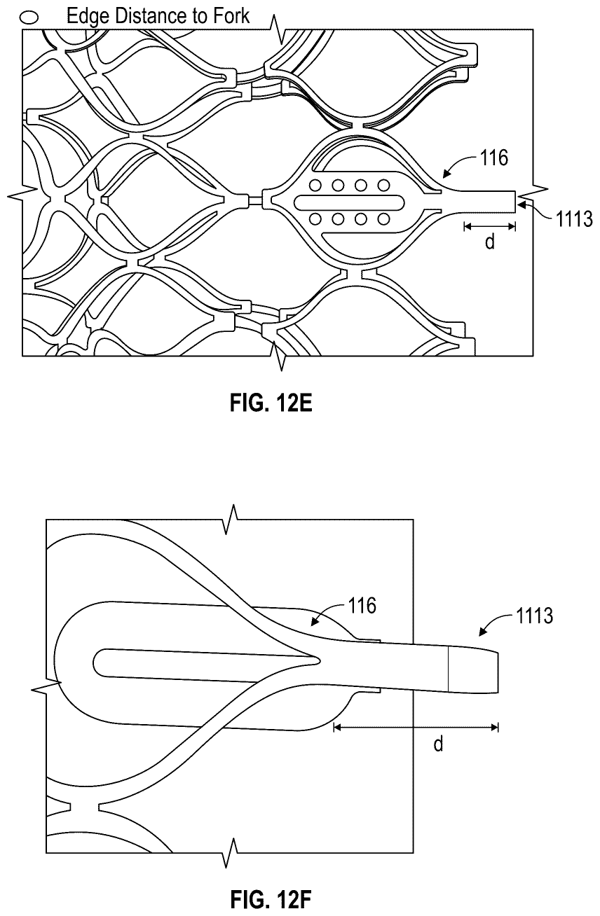

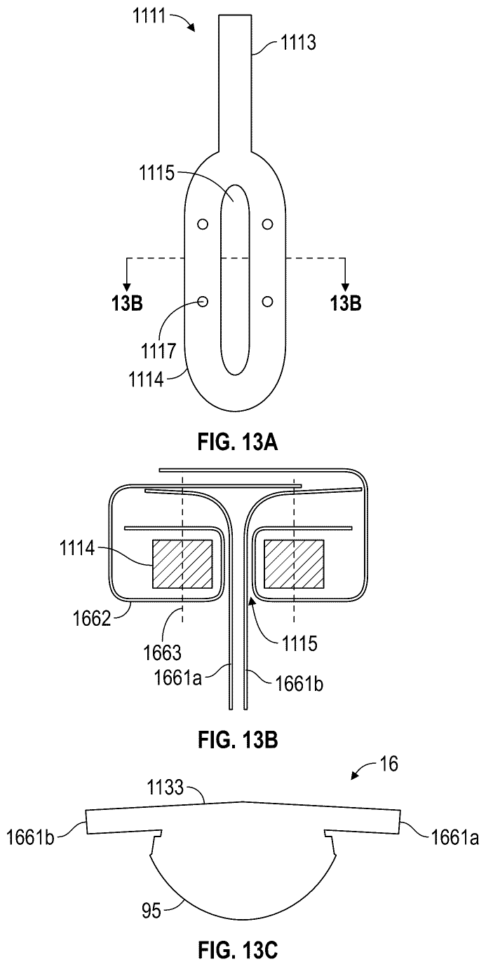

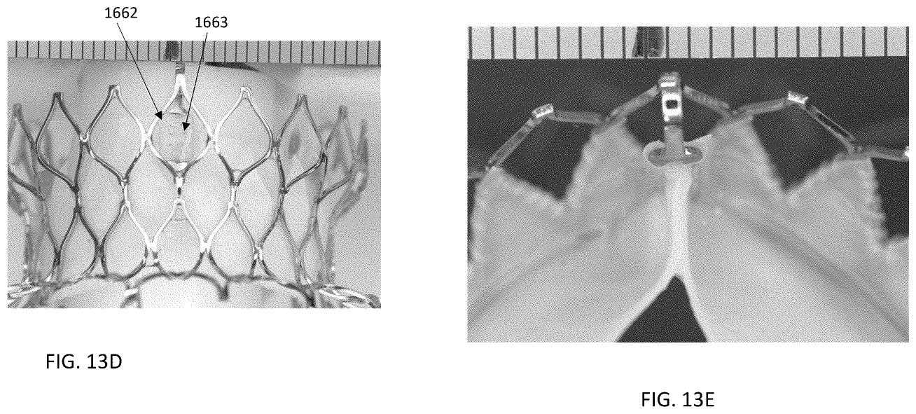

Referring to FIGS. 12A-12D, and particularly to FIG. 12D (which shows a close-up of the attachment mechanisms 1111), the interior commissure attachment mechanisms 1111 can each include a distal post 1113 that extends continuously from the distal end of a distal arch (e.g., crown, or apex) 116 (i.e., the distal-most portion of a cell 122) of the frame structure 12a and curves radially inwards, such as by approximately 180 degrees. The commissure attachment mechanisms 1111 can be integrally formed with the valve frame 12, which provides a number of advantages, including simplifying the manufacturing process, increasing mechanical strength, reducing wear, and requiring no separate attachment mechanism. As shown in FIGS. 12E-12F, the distal post 1113 can have a distance d from the distal end of distal arch 116 until where the distal post curves radially inwards. In some embodiments, this distance d can be approximately 2.5 mm long +/−0.25 mm. In other embodiments, this distance d can range from 2-4 mm. The commissure attachment mechanisms 1111 can further each further include a rounded paddle 1114 (e.g., continuous with the post 1113) that includes a slot 1115 therethrough configured to pass a portion of the leaflets 16 (e.g., tabs 1661 shown in FIG. 13C) therethrough. The slot 1115 can be surrounded by a plurality of sutures holes 1117 configured to enable sewing attachment of the leaflets 16 to the attachment mechanisms 1111.

As shown in FIGS. 13A-13C, each of the leaflets 16 can include an inflow edge 95, and a tab 1661 at each commissure extending radially and parallel with the outflow (e.g., free) edge 1133. To attach the leaflets 16 to the attachment mechanisms 1111, the tabs 1661 can be passed through the slot 1115, folded around the exterior of the paddle 1114, and sewn thereto with a suture 1663 passing through the suture holes 1117. Referring to FIGS. 13B-C, a first tab 1661a of a leaflet 16 can pass through a slot of a first attachment mechanism, and a second tab 1661b can pass through a slot of a second attachment mechanism. Each attachment mechanism is configured to receive a single tab of two adjacent leaflets (e.g., tab 1661a from a first leaflet and tab 1661b from a second leaflet). It should be understood that, when looking at FIG. 13B, two adjacent leaflets are attached to the tab (not two tabs from the same leaflet). In some embodiments, an additional layer 1662 of fabric can be positioned between the tabs 1661 and the paddle 1114 to provide a cushion and/or to prevent rubbing of the leaflets 16 against the paddle 1114. This additional piece of fabric can optionally be wrapped around the paddle 1114 and be secured on the other side with the suture, as shown. FIG. 13D shows overlap of the additional layer 1662 of fabric on the back side of the attachment mechanisms 1111, which is sewn onto the attachment mechanism 1111 with suture 1663. FIG. 13E shows a view looking from the outflow edge at the leaflets 16 where they attach to the attachment mechanism 1111 and the additional layer 1662 of fabric.

In some embodiments, the commissure attachment mechanisms 1111 can have a width or thickness that is greater than the width or thickness of the cell strut or distal arch 116 or other cells 122 of the frame structure 12a. In some embodiments, this increased width or thickness of the commissure attachment mechanism 1111 can help ensure that the attachment mechanism 1111 stays substantially rigid and/or unbending even during opening and closing of the leaflets 16. In other embodiments, the increased width or thickness of the attachment mechanism 1111 can be configured to enable slight bending, thereby distributing strain between the commissure attachment mechanism 1111 and the frame 12a.

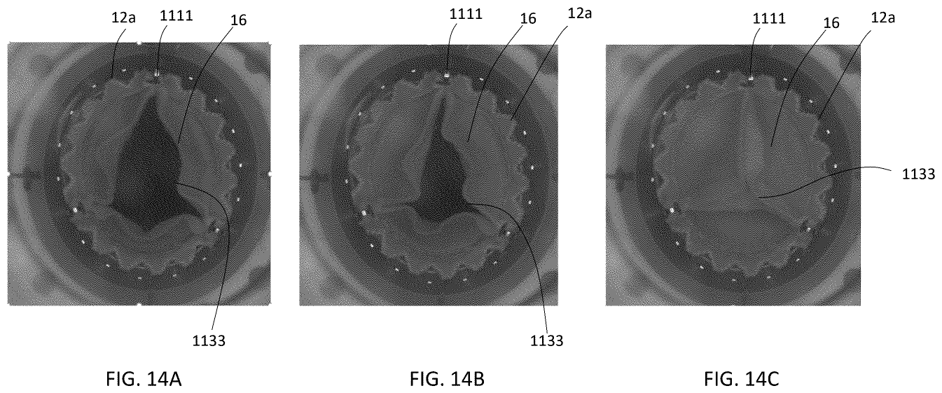

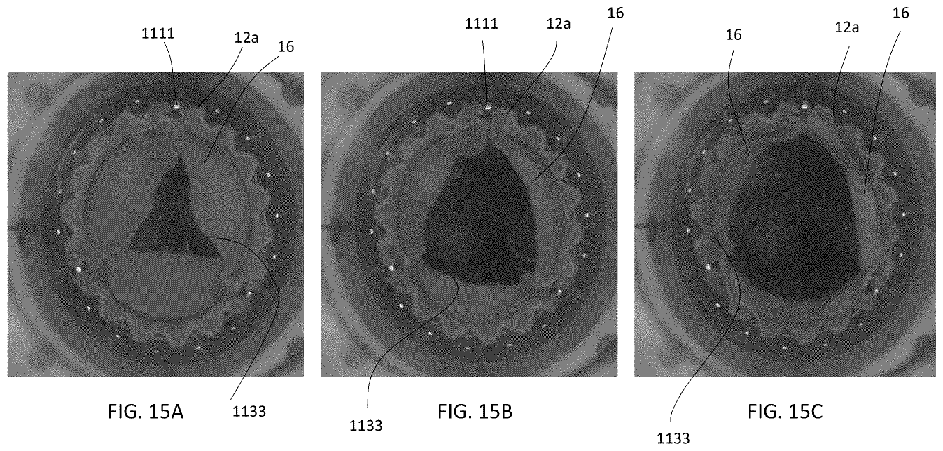

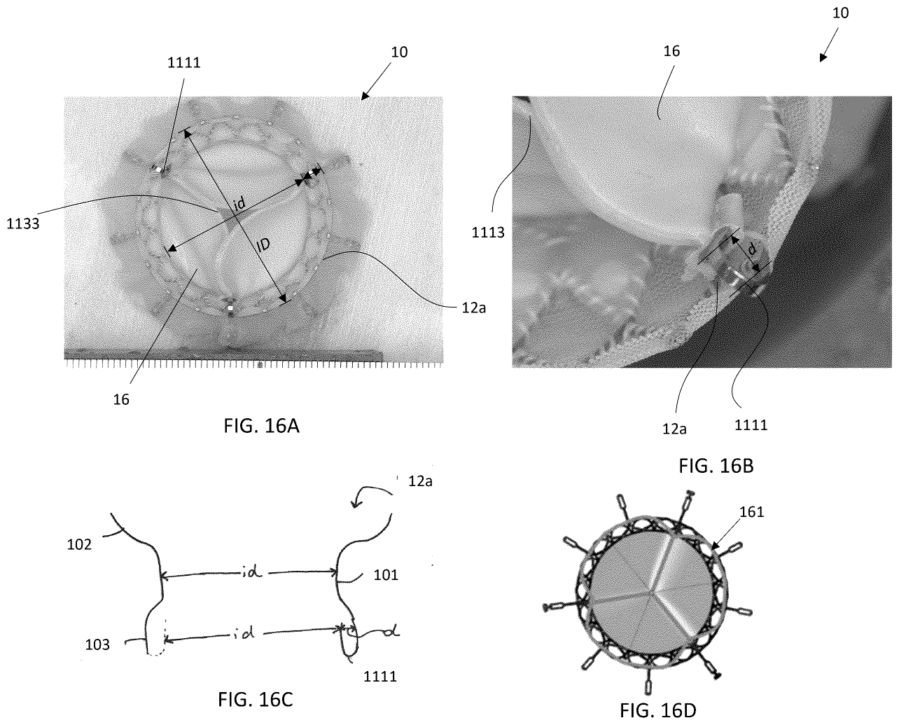

As shown in FIGS. 14A-14C (leaflets closing) and 15A-15C (leaflets opening), the interior commissure attachment mechanisms 1111 can hold the outflow edge 1133 of the leaflets 16 away from the inner perimeter (e.g., diameter) of the frame structure 12a during both opening and closing of the leaflets 16. Referring to FIGS. 16A-16C, for example, the interior commissure attachment mechanisms 1111 can hold the outflow edge 1133 of the leaflets 16 a distance d away from the interior diameter ID of the frame 12a, such as 1.5 mm-4 mm away, such as 2-3 mm, such as approximately 2.5 mm away. Because the outflow edge 1133 of the leaflets 16 is held away from the inner diameter of the frame 12a during both opening and closing of the leaflets 16, deformation to the leaflets 16 is minimized even when the frame structure 12a is deformed (e.g., upon implantation). Additionally, the spacing between the leaflets 16 and the inner diameter of the frame structure 12a can advantageously enable more blood to flow through that area while the leaflets 16 are opening to help prevent blood stagnation between the frame 12a and the leaflets 12a. Finally, the spacing between the leaflets 16 and the inner diameter of the frame structure 12a can help maintain durability of the leaflets 16 (i.e., prevent frictional wear of the leaflets 16 over time). In some embodiments, the leaflets are configured to open to a radius that is greater than a radius of the commissure attachment mechanism but less than a radius of the diameter or perimeter of the frame structure.

As a result of the interior commissure attachment mechanism 1111, the attached edges of the leaflets 16 can extend substantially straight down from the central annular portion 101 (i.e., without curving radially outwards). The free (e.g., outflow) edge of the leaflet (e.g., between commissure attachment mechanisms 1111) can open to a radius that is greater than id, without contacting frame 12a. That is, as shown in FIGS. 16A-16D, the inner diameter id and/or circumference formed by the commissure attachment mechanisms 1111 can be approximately equivalent to the inner diameter id and/or circumference of the frame 12a at the central annular portion 101. Thus, the leaflets 16 can form a substantially cylindrical flow path for blood passing therethrough. In FIG. 16D, an outline 161 of the leaflet material is shown when opened.

Specifically, an offset allows for increased expansion of the leaflet (into the “gap”), without contacting the frame. This increases blood flow space, without deforming leaflet attachment (from that cylindrical profile).

In some embodiments described herein, the inflow edge 95 of the leaflets can be entirely unsupported except at commissures of the leaflets 16, such as except for at the interior commissure attachment mechanism 1111 and/or the minimal valve supports 124.

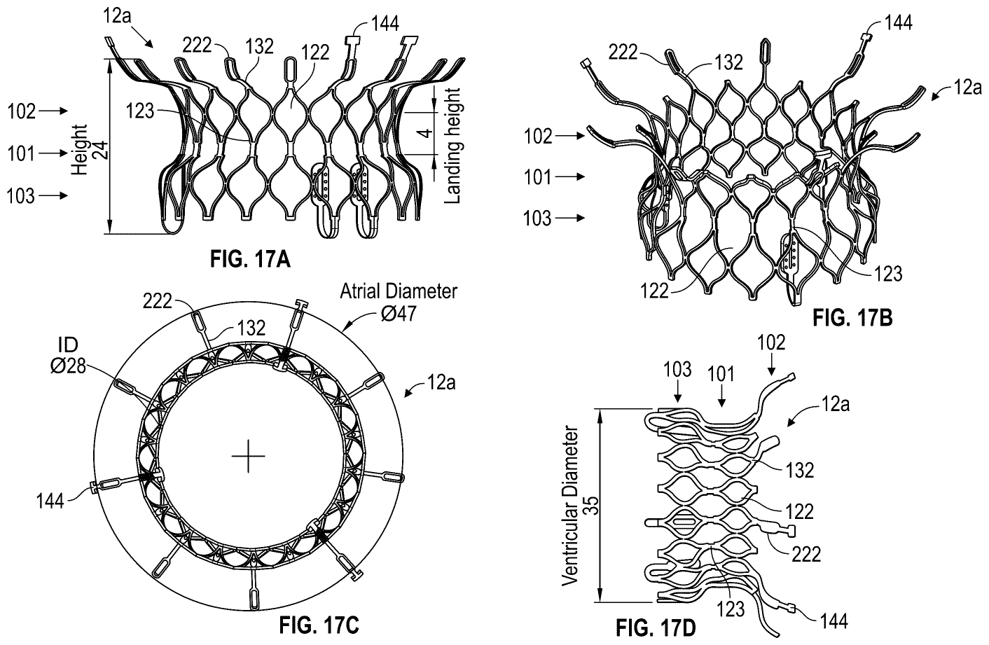

FIGS. 17A-17D show further details of the frame 12a. The frame 12a can include a plurality (e.g., three) rows of cells 122 such that the first row of cells is in the atrial flared portion 102, the second row in the central annular portion 101, and the third row of cells in the ventricular flared portion 103. The cells 122 can be substantially diamond shaped and thus can foreshorten during deployment (e.g., expansion). Further, the row of cells 122 in the central annular portion 101 can include axially extending narrowed struts 123 therein that are non-foreshortening. Additionally, the atrial flared portion 102 of frame 12a can include a plurality of eyelets 222 (e.g., oval eyelets) extending from the vertex of some or all of the atrial cells 122 (for example, from every other vertex as shown in FIGS. 17A-17D). The eyelets 222 can be configured to remain above (or proud of) other portions of the atrial flared portion 102 when implanted and can advantageously enable grasping and/or manipulation of the valve frame 12 (e.g., with a hook or suture) during deployment. The eyelets 222 can be connected to the arches (or vertices) of the cells 122 via a straight extension 132. The straight extension 132 can be non-foreshortening during deployment, which can advantageously help with trackability and sheathing of the atrial flared portion 102 during delivery of the frame 12a. The non-foreshortening extensions 132 can additionally advantageously provide increased apposition on the atrial side, allowing for a reduction in length of the foreshortening elements (e.g., the diamond-shaped cells 122) and thus increasing radial stiffness of the valve frame 12a. In some embodiments, the length of the non-foreshortening extensions 132 can be selected to match the size of the atrial brim of the frame with a given anatomy at an implantation site. The base valve structure can remain the same even when the length of these extensions is adjusted. This creates the ability to provide frames with a plurality of different atrial brim sizes while the rest of the frame has consistent dimensions. The frame 12a can further include a plurality of (e.g., three) frame tabs 144 extending from the eyelets 222 and configured to engage with a valve delivery system for deployment. In one embodiment, each of the frame tabs 144 can be spaced apart by 120°. As shown in FIG. 17A, the frame 12a can have an overall height of 24 mm and a landing height (e.g., “landing zone”, height within which the anchor is configured to rest) of 4 mm. As shown in FIG. 17D, the frame 12a can have a ventricular diameter of 35 mm.

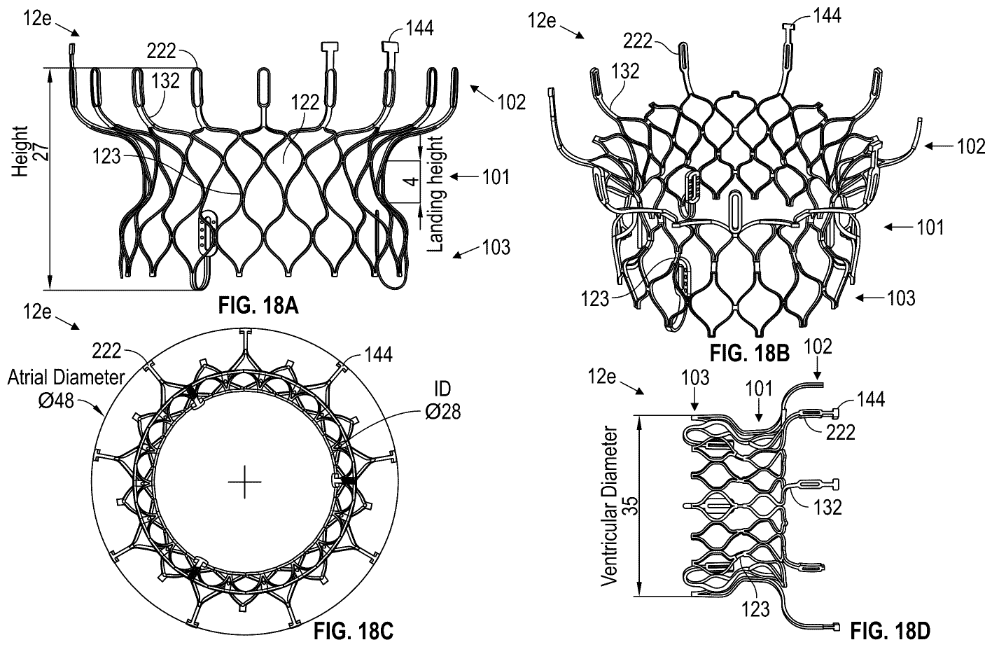

Another exemplary frame 12e is shown in FIGS. 18A-18D. The frame 12e is similar to frame 12a except that frame 12e includes four rows of cells 122. The rows of cells 122 can be configured such that there is an extra row of cells in the atrial flared portion 102 (i.e., such that that there is one row of cells in the ventricular flared portion 103, one row of cells in the central annular portion 101, and two rows of cells in the atrial flared portion 102). Additionally, the cells 122 in the atrial flared portion 102 can be flared further radially outwards in the frame 12e than the frame 12a so as to better engage with the atrial wall proximate to the annulus. Further, the straight extensions 132 can be curved so as to point the eyelets 222 substantially in the axial direction (i.e., towards the atrium). The additional row of cells 122 on the atrial side can advantageously increase the stiffness of the atrial flared portion 102, thereby helping to anchor the atrial flared portion 102 in the atrium (and/or pull the anchor up towards the annulus upon deployment of the frame 12e). Frame 12e can have an overall height of 27 mm and a landing height of 4 mm. As shown in FIG. 18D, the frame 12e can have a ventricular diameter of 35 mm.

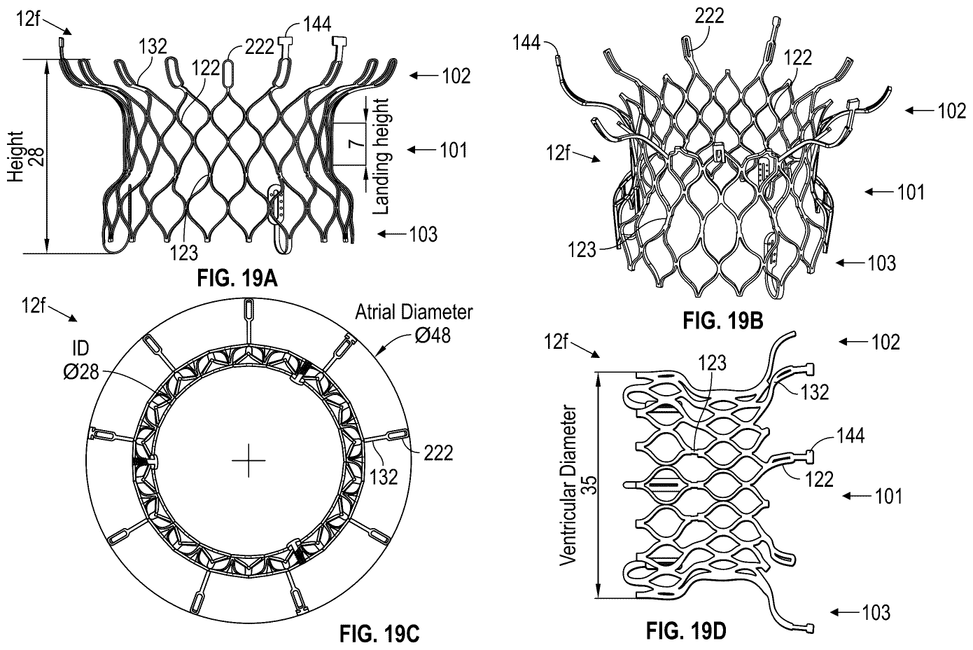

Another exemplary frame 12f is shown in FIGS. 19A-19D. The frame 12f is similar to frame 12e except that the additional row of cells 122 at the atrial end is flared less radially outwards (i.e., the vertices can point more in the axial direction rather than outwards in the radial direction). Reducing the amount of flaring can increase the landing zone height, which can better enable the anchor 15 to be positioned against the central annular section 101. Frame 12f can have an overall height of 28 mm and a landing height of 7 mm. As shown in FIG. 19D, the frame 12f can have a ventricular diameter of 35 mm.

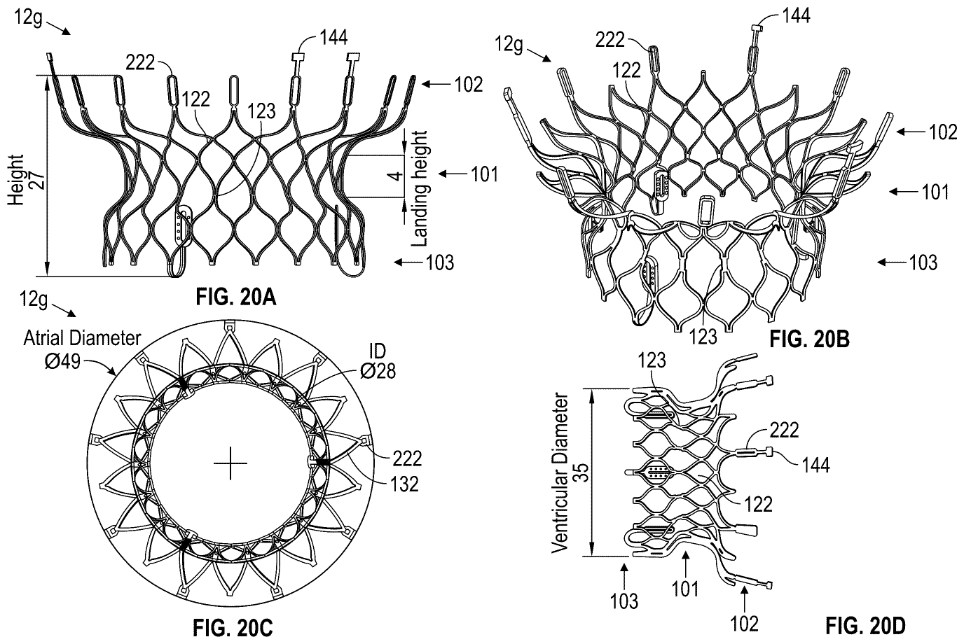

Another exemplary frame 12g is shown in FIGS. 20A-20D. The frame 12g is similar to frame 12e except that the atrial-most cells 122 are lengthened so as to extend up to the axial location of the eyelets 222 (eliminating straight extensions 132). This increase in length of the atrial-most cells 122 can result in the vertices pointing more axially, thereby making the vertices less traumatic. Like frame 12e, the frame 12g can have an overall height of 27 mm and a landing height of 4 mm. As shown in FIG. 20D, the frame 12g can have a ventricular diameter of 35 mm.

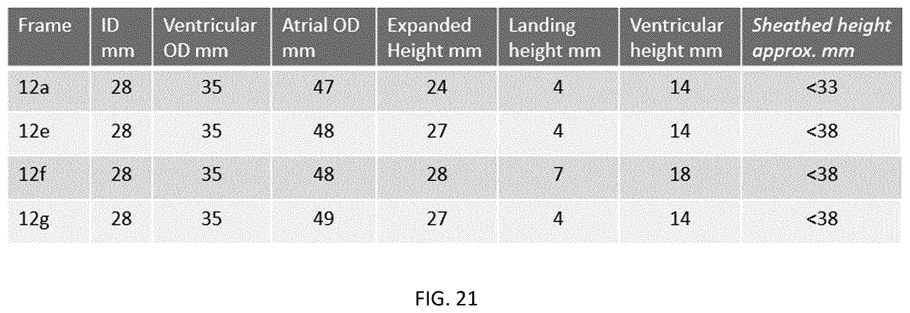

A table showing exemplary dimensions for frames 12a, 12e, 12f, and 12g is shown in FIG. 21. Referring to the table of FIG. 21, all of the frames can have an ID of 28 mm and a ventricular OD of 35 mm. However, the atrial OD is adjusted between the frames, ranging from 47 mm for the 12a frame up to 49 mm for the 12g frame. The expanded height can also vary between the frames, ranging from 24 mm for frame 12a, 27 mm for frame 12e and 12g, and 28 mm for frame 12f. The landing height of the frames can be about 4 mm for the 12a, 12e, and 12g frames, and about 7 mm for frame 12f. The ventricular height can be about 14 mm for frames 12a, 12e, and 12g, and about 18 mm for frame 12f. Frame 12a can have a sheathed height of less than 33 mm, while frames 12e, 12f, and 12g can have sheathed heights of less than 38 mm.

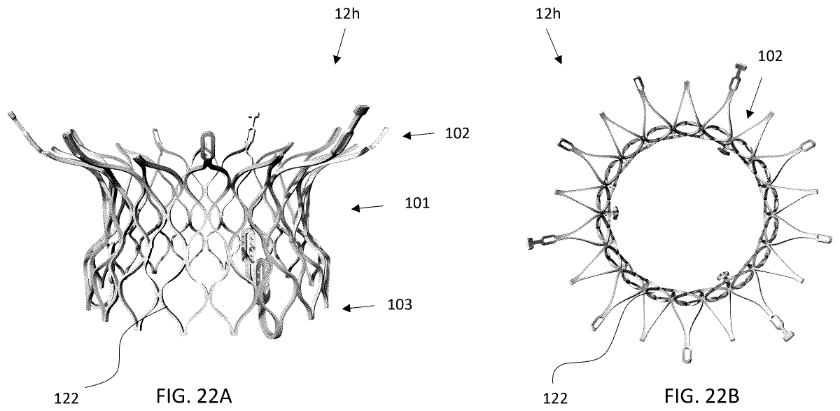

Another exemplary frame 12h is shown in FIGS. 22A-22B. The frame 12h is similar to frame 12g except that the atrial flared portion 102 flares further outward to a larger radius. Additionally, the distal-most cells 122 are more elongate. Finally, the landing height can be slightly larger at approximately 4-5mm.

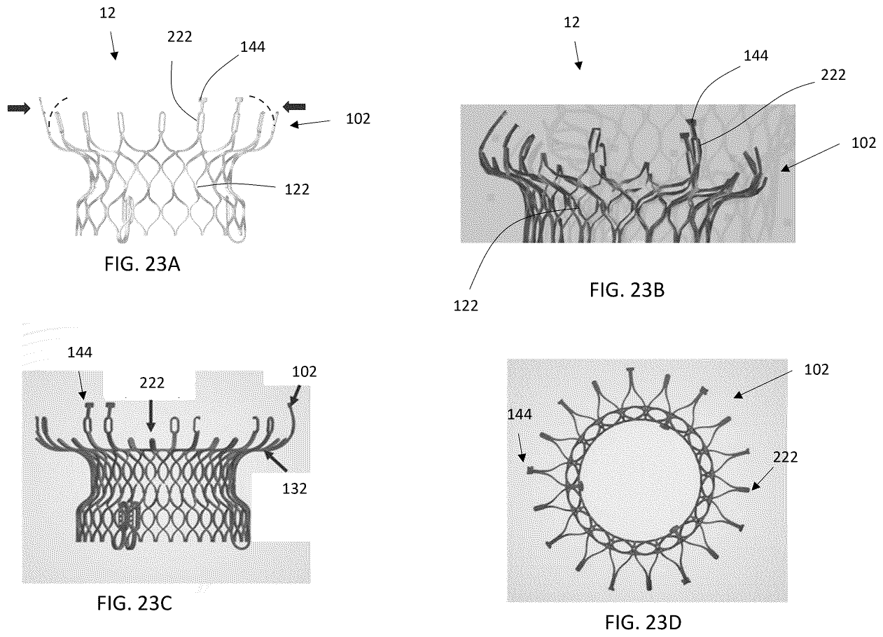

Referring to FIGS. 23A-23B, any of the frames 12 described herein (e.g., frames 12a-12h) can be modified such that the atrial flared portion 102 curves around (e.g., in a C-shape) such that the distal tips point radially inwards. Doing so can help reduce trauma to the atrium when the valve prosthesis is implanted. In some embodiments, at least some of the distal tips can point inwards with a radius of curvature below 10 mm. In another embodiment, the radius of curvature of the distal tips can be between 3-10 mm. In some embodiments, as shown in FIGS. 23C-23D the inward curvature of the atrial flared portion 102 can be formed by keeping the foreshortening cells 122 the same dimension, but increasing the length of the non-foreshortening elements on the atrial side (e.g., the straight extension 132, eyelets 222, or frame tabs 144) and curving those non-foreshortening elements upward and/or inwards.

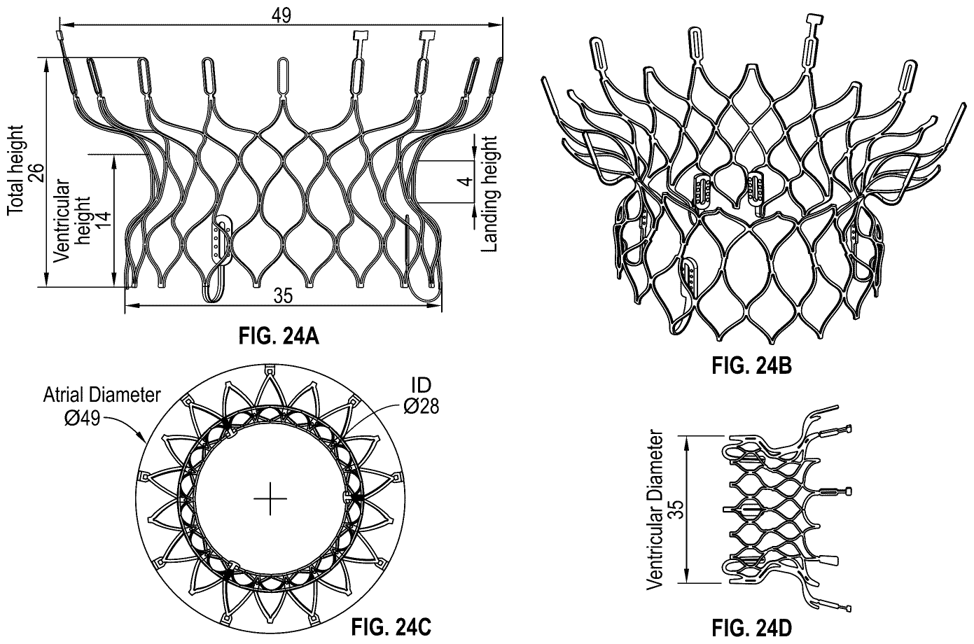

FIGS. 24A-24D illustrate another frame embodiment with a total height of 26 mm, a ventricular height of 14 mm, a ventricular OD of 35 mm and a landing height of 4 mm. This embodiment includes longer atrial struts with no inward curvature and no extended paddle arms compared to some of the embodiments described above.

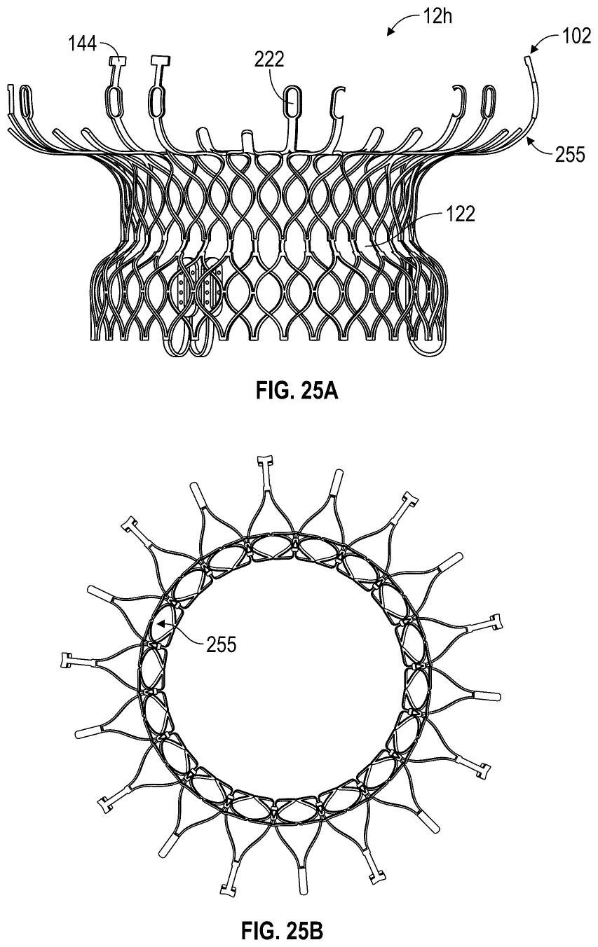

FIGS. 25A-25B illustrate another embodiment of a frame 12h that includes a pattern of cells 122 that terminate in a horizontal section 255 of the frame near the atrial portion 102. This allows for easier variation of the atrial brim size depending on the specific need of a particular patient. For example, in the frame 12h of FIGS. 25A-25B can have an atrial OD of 53 mm. In another embodiment, the length of the cells on the atrial side of the frame, such as the cells that terminate at or in the horizontal section 255, can be longer than the other cells to increase the atrial diameter of the frame.

Any of the valve frames 12 (e.g., frame 12a-12h) described herein can include one or more skirts or seals thereon. For example, the valve frame 12 can include an internal skirt and one or more external skirts. The skirts can be made, for example, of PET.

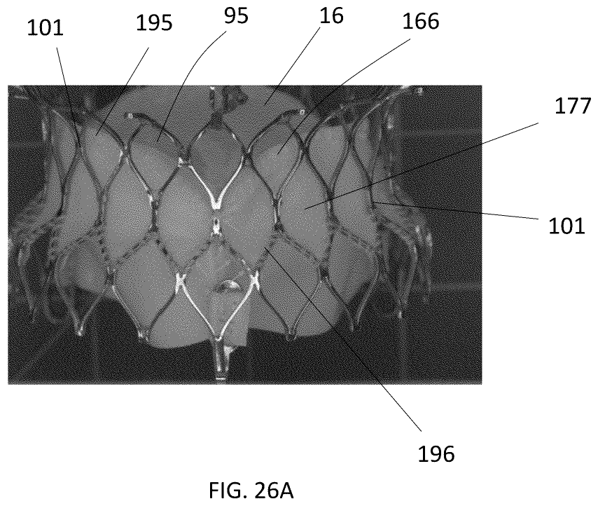

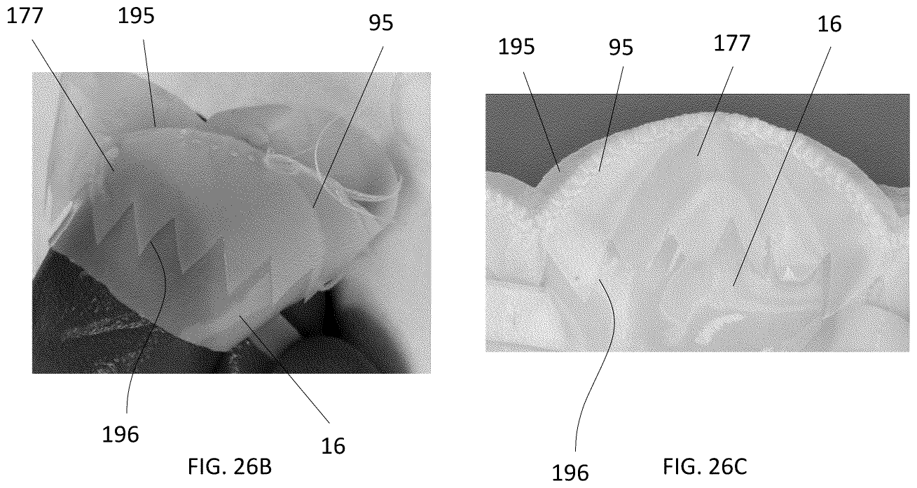

Referring to FIGS. 26A-26C, an internal skirt 177 can be attached to the internal circumference of the frame 12. The inflow edge 195 of the internal skirt 177 can include three convex segments 166 configured to at least partially conform to the inflow edges 95 of the leaflets 16. In some embodiments, as shown in FIGS. 26B-26C, the radius of curvature of the convex segments 166 of the inflow edge 195 of the internal skirt 177 can be greater than the radius of curvature of the inflow edges 95 of the leaflets 16. Further, the internal skirt 177 (e.g., the inflow edges 195) can be attached to the inflow edges 95 of the leaflets 16. The internal skirt 177 may thereby (e.g., indirectly) couple the inflow edges 95 of the leaflets 16 to the frame 12. The outflow edge 196 of the internal skirt 177 can be cut in a pattern (e.g., a zigzag or repeating triangular pattern) configured to match the cell pattern of the frame 12. The outflow edge 196 can be attached to the frame 12 within and/or distal to the central circumferential portion 101, and proximate to the outflow region 103. Thus, the internal skirt 177 may not extend all the way to the ventricular end of the frame 12.

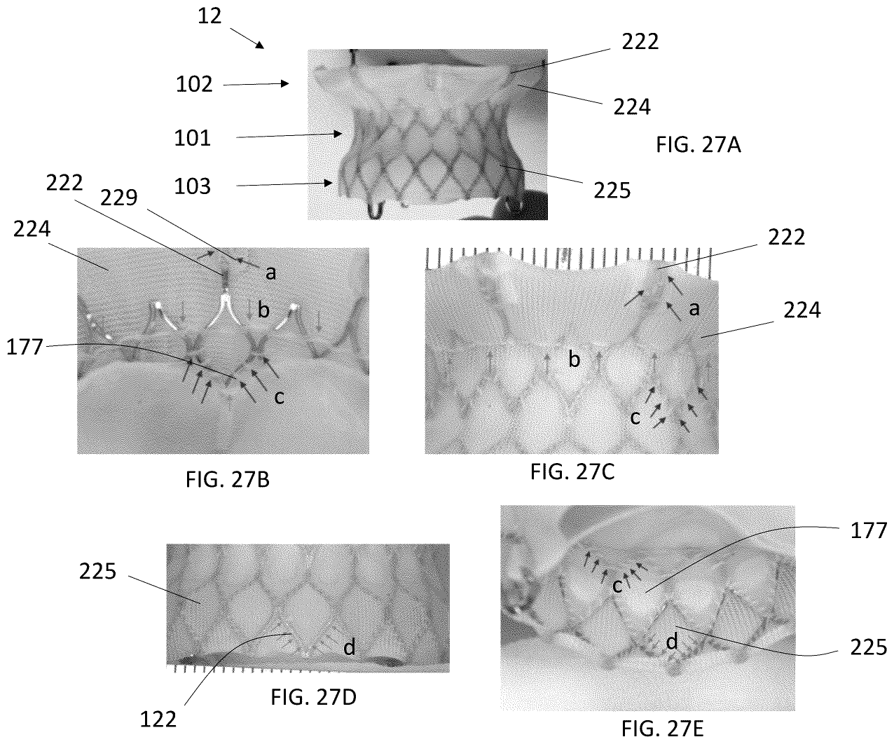

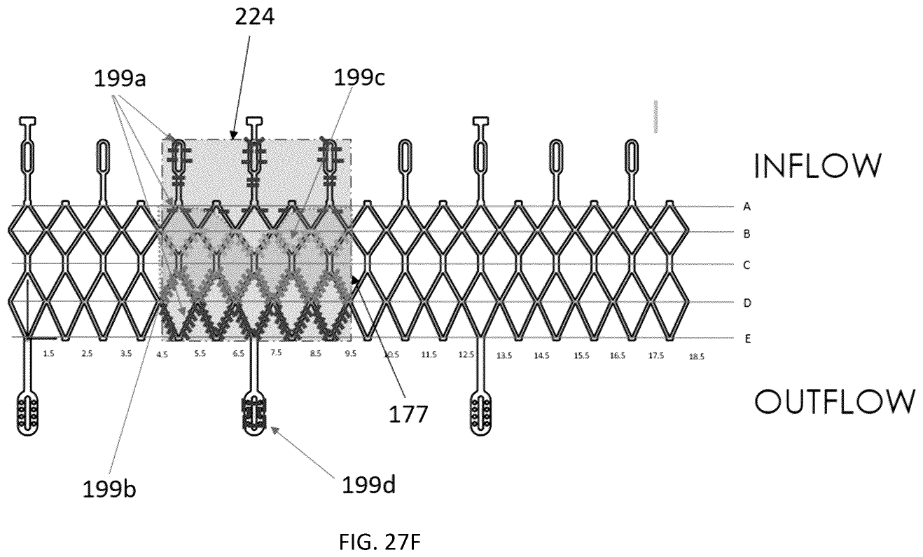

Referring to FIGS. 27A-27F, in some embodiments, the ventricular flared portion 103 can further include an external skirt 225 thereon. The ventricular external skirt 225 can advantageously prevent chordae from interacting with (e.g., getting stuck in) the cells and/or tips of the ventricular flared portion 103, and/or can aid in making the ventricular flared portion 103 less traumatic. The external skirt 225 can, for example, wrap around the ventricular end of the frame 12 so as to cover the exposed vertices of the cells 122.

FIGS. 27A-27F also show exemplary frame attachment locations for the internal skirt 177, the atrial external skirt 224, and the ventricular external skirt 225. As best shown in FIGS. 27B, 27C, and 27D, the inner skirt 177 can be sewn to the frame 12 proximate to the tips of the atrial flare (shown by the arrows c pointing to the stitches). As shown best in FIGS. 27B-27C, the atrial external skirt 224 can be attached to the frame 12 such that the skirt tabs 229 fold over (and are sewn to) the eyelets 222 (shown by arrows a pointing to the stitches). Additionally, the atrial external skirt 224 can be sewn around the circumference of the frame 12 near the tips of the flare (shown by the arrows b pointing to the stitches). As best shown in FIGS. 27D-27E, the ventricular external skirt 225 can be attached such that the edge of the skirt 225 folds over the tips of the ventricular flare. Additionally, the ventricular external skirt 225 can be sewn to the frame 12 along the distal (ventricular)-most struts of the frame 12 (shown by the arrows d point to the stitches). In some embodiments, the external skirts 224, 225 can overlap one another proximate to the central annular circumferential portion 101. In other embodiments, the external skirts 224, 225 can have an axial space therebetween, e.g., such that a central section (e.g., the central annular portion 101) does not have an external skirt thereon.

Referring to FIG. 27F, the frame can include an internal skirt 177 and an external skirt 224. The external skirt 224 can be sewn to the frame at attachment points 199a. The internal skirt 177 can be sewn to the frame at attachment points 199b. Additionally, both the external skirt and the internal skirt can be sewn to the frame at attachment points 199c. Additionally, as described above, the commissure attachment mechanisms can include attachment points 199d for sewing attachment to a fabric strip, tissue, and the frame.

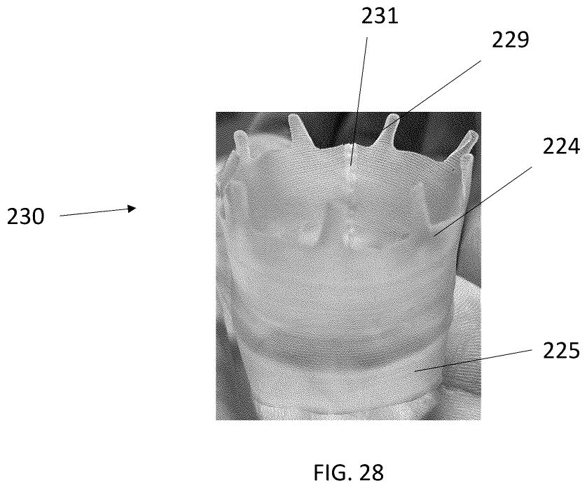

Referring to FIG. 28, in some embodiments, the atrial and ventricular external skirts 224, 225 can be part of a single unitary skirt 230. The single unitary skirt 230 can advantageously help prevent bunching and/or folding that may occur with two separate skirts. In some embodiments, the single unitary skirt 230 can be manufactured from a flat sheet of knitted material that is laser cut and sewn together (e.g., along seam 231). In this embodiment, the cylindrical single unitary skirt 230 can then be dip coated and shape set over a mandrel. In other embodiments, the single unitary skirt 230 can be manufactured from a tube knit fabric. In this embodiment, the tube knit fabric can be slid over a mandrel, dip coated, and then laser cut into a pattern while still positioned on the mandrel. This embodiment can advantageously form a seamless single unitary skirt 230.

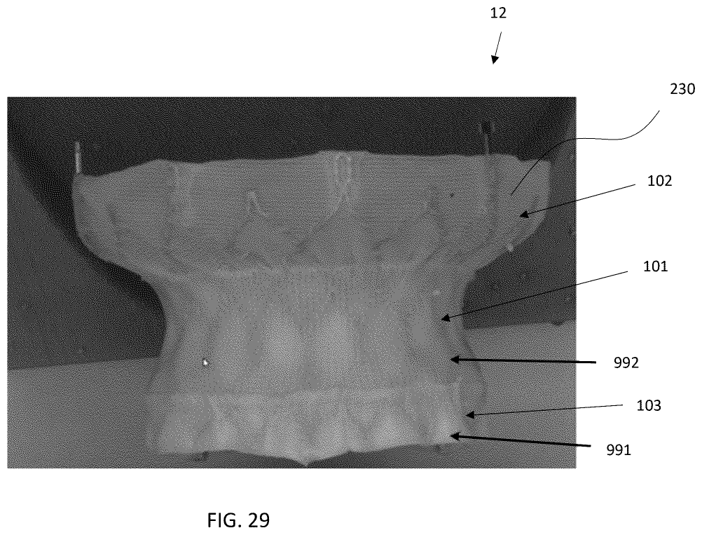

Referring to FIG. 29, in some embodiments, the frame can include a unitary skirt 230 (e.g., as described with respect to FIG. 28) with additional layers along a portion of the frame 12. For example, the single unitary skirt 230 can be layered with an additional ventricular skirt section 991 positioned along the distal-most struts of the frame 12 and/or folded thereover. The additional ventricular skirt section can advantageously help reduce trauma to the native valve (e.g., to the chordae). The single unitary skirt 230 can additionally or alternatively be layered with an additional skirt section 992 at the central annular section 101, which can help prevent damage to the native leaflets.

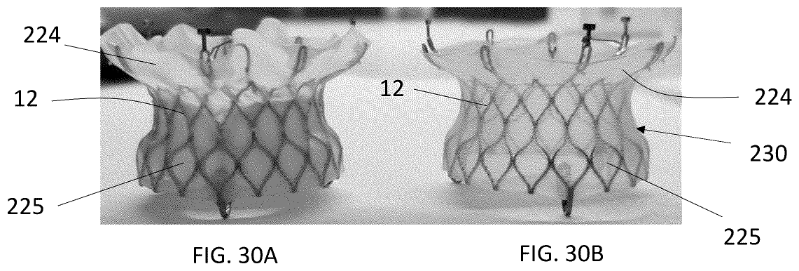

The skirts described herein can be made of a polymer, such as polyethylene terephthalate (PET). Further, the skirts described herein can be woven and/or knitted (e.g., with a denier of 15-25, such as approximately 20). For example, FIG. 30A shows a frame 12 having a woven atrial external skirt 224 and a knitted ventricular external skirt 225. In contrast, FIG. 30B shows a frame 12 having a knitted unitary skirt 230.

Any of the skirts described herein can include a coating, such as a chronoflex AR coating, to reduce the pore size of the skirt and increase resistance to fluid flow therethrough. For example, in some embodiments, only the external atrial skirt 224 can include a coating thereon. In other embodiments, both the external and atrial skirts 224, 225 (either individual or unitary) can include a coating thereon.

As described herein, the valve prosthesis may include a frame structure (e.g., with a tapered waist and atrial and ventricular flared portions) with leaflets therein. In some embodiments, the leaflets can be formed of multi-layered materials for preferential function. The leaflets may be attached directly to the frame structure. Alternatively, the leaflets may be attached to an intermediate valve structure that is in turn connected to the frame structure. The leaflets may be connected to the frame structure before or after the frame structure has been deployed adjacent a native valve. The leaflets may comprise a biocompatible one-way valve. Flow in one direction may cause the leaflets to deflect open and flow in the opposite direction may cause the leaflets to close. The frame structure may be configured like a stent. The frame structure may, for example, comprise a scaffold in a diamond pattern formed from a shape memory material (e.g., nitinol, NiTi). One of ordinary skill in the art will appreciate that many other structures, materials, and configurations may be employed for the frame structure. For example, the frame structure may be formed of a polymer of sufficient elasticity. The frame structure may be formed of a combination of metal and polymer, such as metal (e.g., shape memory material) covered in polymer. The frame structure may include a variety of patterns besides diamond shapes. In some embodiments, the frame structure is a closed frame such that blood flow is forced through the leaflets therein. One or more skirts and/or seals may help force blood through the leaflets. Exemplary frame structures and valve prostheses are described in PCT Application No. PCT/US2019/047542, filed Aug. 21, 2019, titled “PROSTHETIC CARDIAC VALVE DEVICE, SYSTEMS, AND METHODS,” now PCT Publication No. WO 2020/041495 in International Patent Application No. PCT/US2020/027744, filed Apr. 10, 2020, titled “MINIMAL FRAME PROSTHETIC CARDIAC VALVE DELIVERY DEVICES, SYSTEMS, AND METHODS,” now PCT Publication No. WO 2020/210685, and in International Patent Application No. PCT/US2021/037661, filed Jun. 16, 2021, titled “MINIMAL FRAME PROSTHETIC CARDIAC VALVE DELIVERY DEVICES, SYSTEMS, AND METHODS,”the entireties of which are incorporated by reference herein.

Additionally, in some embodiments, the valve prostheses described herein include one or more anchors. The anchor may include a flat spiral shape with a plurality of windings or loops spiraling radially outwards from a central point. The loops of the flat spiral shaped anchor may be generally positioned within the same plane. The anchor may be formed from a shape memory material (e.g., NiTi). The anchor can be configured to extend around the chordae of the valve (e.g., the mitral valve) and around the valve prosthesis to hold the valve prosthesis in place. Flat spiral anchors are described in U.S. patent application Ser. No. 16/723,537, filed Dec. 20, 2019, titled “PROSTHETIC CARDIAC VALVE DEVICES, SYSTEMS, AND METHODS,” now U.S. Publication No. US-2020-0261220-A1, the entirety of which is incorporated by reference herein.

The valve prostheses and/or anchors described herein may be delivered via a delivery system. Exemplary delivery systems are described in International Application No. PCT/US2020/023671, filed Mar. 19, 2020, titled “PROSTHETIC CARDIAC VALVE DEVICES, SYSTEMS, AND METHODS,” now PCT Publication No. WO 2020/191216, and in International Application No. PCT/US2021/040623, filed Jul. 7, 2021, titled “VALVE DELIVERY SYSTEM,”the entireties of which are incorporated by reference herein.

It should be understood that any feature described herein with respect to one embodiment can be substituted for or combined with any feature described with respect to another embodiment. For example, it should be understood that any of the frame structures, features of the frame structures (e.g., commissure attachment mechanisms), or skirts described with respect to one embodiment can be interchanged and/or combined with any of the other frame structures described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled”to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. (canceled)

2. A device for treating a diseased native valve in a patient, the device comprising:
a frame structure comprising an annular central portion, a flared inflow portion, and a flared outflow portion;
a valve segment positioned radially within the frame structure, the valve segment comprising a plurality of leaflets; and
an internal skirt attached to the frame structure, the internal skirt comprising a plurality of convex segments configured to at least partially conform to inflow edges of the leaflets.

3. The device of claim 2, wherein the convex segments have a greater radius of curvature than the inflow edges of the leaflets.

4. The device of claim 2, wherein an outflow edge of the internal skirt comprises a zig-zag pattern configured to match a cell pattern of the frame structure.

5. The device of claim 2, wherein an outflow edge of the internal skirt is attached to the frame proximate to the annular central portion.

6. The device of claim 2, wherein an outflow edge of the internal skirt does not extend to an outflow end of the frame structure.

7. The device of claim 2, wherein the inflow edges of the leaflets are unsupported by the frame structure.

8. The device of claim 2, wherein the inflow edges of the leaflets are spaced radially inwards from an inflow end of the frame structure.

9. The device of claim 2, further comprising a spiral anchor configured to be placed around the frame structure.

10. A device for treating a diseased native valve in a patient, the device comprising:
a frame structure comprising an annular central portion, a flared inflow portion, and a flared outflow portion;
a valve segment positioned radially within the frame structure, the valve segment comprising a plurality of leaflets; and
an external skirt attached to the frame structure, the external skirt comprising a unitary structure covering the flared inflow portion and the flared outflow portion.

11. The device of claim 10, wherein the external skirt comprises a tube knit fabric.

12. The device of claim 10, wherein the external skirt comprises a coating thereon.

13. The device of claim 10, further comprising a spiral anchor configured to be placed around the frame structure.

14. The device of claim 10, wherein the external skirt is wrapped over an outflow edge of the frame structure.

15. The device of claim 10, further comprising an additional skirt layered with the external skirt.

16. The device of claim 15, wherein the additional skirt is positioned along the flared outflow portion.

17. The device of claim 10, wherein the additional skirt is positioned along a central annular portion of the frame structure.
